(19) United States
(12) Patent Application Publication
(10) Pub. No.: US 2018/0000353 A1
(43) Pub. Date: Jan. 4, 2018

(54) SYSTEM FOR DETECTING THE VITAL STATUS OF ANIMALS

(71) Applicants: Andreas THIEME, Pulsnitz (DE); Torsten URSINUS, Dresden (DE)

(72) Inventors: Andreas THIEME, Pulsnitz (DE); Torsten URSINUS, Dresden (DE)

(21) Appl. No.: 15/602,090

(22) Filed: May 22, 2017

(30) Foreign Application Priority Data
Jun. 30, 2016 (DE) 20 2016 004 112.7

(51) Int. Cl. A61B 5/0205; A61B 5/00; A61B 5/11; A61B 5/01; A61D 3/00; A61B 5/1455; A61B 5/024; A61B 5/053

(52) U.S. Cl. CPC A61B 5/0205; A61B 5/01; A61B 5/6823; A61D 3/00; A61B 5/1112; A61B 5/112; A61B 5/0002; A61B 5/02438; A61B 5/053; A61B 2503/40; A61B 2560/0252; A61B 2562/06; A61B 5/14552

(57) ABSTRACT

The invention relates to a system for acquiring the vital status of animals, which comprises at least one first device for acquiring vital functions, and at least one signal transmission means, wherein the first device has at least two first and/or second sensors arranged at a distance from each other. The components of the system for acquiring the vital status of animals can be detachably connected with each other by connectors.

The invention further relates to a method for determining the vital status of animals, wherein the vital functions are acquired by a first device with at least two first and/or second sensors arranged at a distance from each other, wherein the acquired data are transmitted by at least one signal transmission means to a data processing device, and there processed and evaluated, and from the data processing device to an external output device, which outputs the vital status of animals.

The invention relates to a system for acquiring the vital status of animals.

The system for acquiring the vital status of animals here comprises various components, such as a first device for acquiring vital functions and at least one signal transmission means. The components of the system for acquiring the vital status of animals are designed so that they can be detachably connected with each other via connectors.

Sensors for GPS tracking or measuring vital functions are currently in collars, harnesses or articles of clothing for animals.

A number of documents describe the determination of positions using GPS technology incorporated in animal harnesses. For example, document DE 20 2011 109 024 U1 discloses a container with a GPS locating device that can be fixed to a dog harness. US 20070204803 A1 describes a GPS transmitter secured to the collar of an animal for position determination.

Document U.S. Pat No. 6,263,836 B1 describes a device for monitoring the behavior of animals, which simultaneously trains and raises the latter through stimulation. The device is here fastened to the harness.

EP 2196086 A1 shows a device for monitoring large animals, such as horses, with a sensor device that measures vital functions like heart rate, body temperature or coat moisture. If limits stored in a memory are exceeded, a signal is transmitted to an external warning device. The device is held against the chest of the horse on a harness comprised of a connecting belt that runs over the stomach, chest and between the legs.

Known from WO 2013/008115 A1 is a system with an animal collar for monitoring the health and vital signs with a warning and diagnosis function. The collar measures body functions, such as respiration, pulse, temperature and movement, and contains a processor that interprets the results. In an embodiment of the invention, the system can also compare its own physiological data with comparative data for the respective animal breed.

DE 10 2014 108 443 A1 discloses a monitoring device for pets and farm animals, especially dogs. The latter comprises a sensor for acquiring the vital functions (e.g., heart and respiration rate, body temperature, blood sugar) of animals, e.g., dogs, cats or horses, and is secured to a carrier device (collar and/or harness and/or halter). The monitoring device further comprises an evaluator that communicates with the sensor. The later also has a receiver separate from the carrier device, to which the measured data are wirelessly transmitted via a WLAN or Bluetooth connection (e.g., via a smartphone).

In most publications, the sensor equipment is most often provided just on a part of the body of the animal, for example on the neck via a collar, and not distributed over the body, disadvantageously making it impossible to guarantee a reliable measurement of vital functions.

Document US 20150181840 A1 describes a collar or harness for animals that contains several sensors. The sensors measure parameters for vital functions, and the data obtained here are either evaluated directly via an evaluator in the collar or harness, or transmitted to a data management server via ultra-wideband technology.

US 2008/0255468 A1 describes a monitoring and/or measuring system for physiological parameters in mammals. Sensors or electrodes for EKG measurement are here fastened in or on articles of clothing or harnesses or pockets or backpacks located thereon at the anatomical locations to be measured. The measured data can either be stored in memory options provided on the article of clothing, or transmitted wirelessly via Bluetooth to evaluation units for real-time measurement.

DE 10 2006 027 764 A describes a heartbeat acquisition system for dogs. A detector for detecting the heartbeats of the animal is here held at the position of the heart by a harness. The detector here exhibits several electrodes, which are arranged on the harness. The harness, which comprises a breast belt, a collar, neck and sternum web, also holds the accompanying data memory.

The object is to provide a system that overcomes the disadvantages of prior art.

A system is here to be provided that guarantees a reliable measurement of the vital functions of the animals in real time by securing the sensors to anatomically suitable and relevant locations of the animal body. In addition, the system is to be capable for application in all animal species.

In addition, the owner of the animal is to be given the ability to monitor both the health status and current position of the animal.

In the invention, the object is achieved by a system according to the independent claim. Advantageous embodiments of the invention are described in the dependent claims.

A first aspect of the invention relates to a system for acquiring the vital status of animals, which according to the invention comprises at least one first device for acquiring vital functions, and at least one signal transmission means, wherein the first device has at least two first sensors arranged at a distance from each other. The vital status here comprises the vital functions of the animal, also referred to as body functions or physiological data.

In an embodiment, the system is designed as system for acquiring the vital status of animals. In an embodiment, the term animal is understood to mean any living creature. In another embodiment, the term animal is understood to mean any vertebrate, which also includes birds. The system for acquiring the vital status is provided exclusively for animals, and not intended for use in humans.

In another embodiment, the term animal is understood to mean any mammal. In another embodiment, the term animal is understood to mean any animal with four legs. An animal with four legs is also referred to as a quadruped. In particular, these also include working and farm animals. The term quadruped further encompasses dogs, cats, horses, in particular racing and sporting horses, and camels.

In the following, the system for acquiring the vital status of animals is abridged to a system for acquiring the vital status.

In an embodiment, the system for acquiring the vital status comprises various components.

The components of the system are advantageously modular in design, and can be replaced, exchanged or retrofitted depending on the application or in the event of repairs or damages. In an embodiment, the system for acquiring the vital status comprises as modules a first device for acquiring vital functions, a module for position determination, orientation and navigation, a module for voice and data transmission, gas sensors, a module for positive owner identification as well as a module for deactivation and blocking.

In an embodiment, the components of the system for acquiring vital functions are advantageously protected against environmental influences, such as shocks, vibrations or extreme temperatures.

In an embodiment, the components of the system for acquiring vital functions are protected against dust according to a military standard (MIL-STD 810).

In an embodiment, the components of the system for acquiring vital functions have at least a first device for acquiring vital functions and at least one signal transmission means.

In an embodiment, the first device for acquiring vital functions has more than two first sensors arranged at a distance from each other, advantageously enabling several measuring points, and hence a more reliable measurement of the vital functions.

In an embodiment, the first sensors acquire data about the vital functions of the animal. In an embodiment, the data about vital functions are here not measured using invasive and percutaneous methods, thereby advantageously preserving the animal and not disrupting its behavior.

Vital functions are understood as vital processes in the waking state, respiration and circulation of the animal, for example the heartrate, blood pressure, pulse rate, respiratory rate and body temperature.

In a preferred embodiment, the at least two first sensors arranged at a distance from each other are designed as a pulse oximeter and/or body temperature sensor and/or temperature sensor and/or pedometer and/or bioelectric resistance sensor.

In an embodiment, the pulse oximeter percutaneously measures the pulse and arterial blood oxygen saturation or oxygen content in the blood of the animal by measuring light absorption or light emission. For this purpose, the at least two pulse oximeters arranged at a distance from each other are advantageously attached along the heart axis positions of the animal.

In an embodiment, the body temperature sensor measures the body temperature of the animal. By measuring the body temperature, it can advantageously be determined whether the animal is undercooled or overheated.

In an embodiment, the temperature sensor measures the ambient temperature of the animal. By measuring the ambient temperature, the still tolerable outside temperature of the animal is advantageously determined, thereby transmitting an early warning in the event of a fire, for example.

In an embodiment, the pedometer measures the number of steps, and thus the distance covered by the animal. In another embodiment, the pedometer is designed as an acceleration sensor (accelerometer).

In an embodiment, the bioelectric resistance sensor measures the body composition of the animal. Body composition includes parameters like body water, fat-free mass, lean body mass, fat mass, body cell mass as well as extracellular mass. In an embodiment, the bioelectric resistance sensor is designed as an impedance sensor.

In a preferred embodiment, the first device further has at least two second sensors arranged at a distance from each other, which are different than the at least two first sensors arranged at a distance from each other. In another preferred embodiment, the selected first sensors and second sensors are a pulse oximeter and/or body temperature sensor and/or temperature sensor and/or pedometer and/or bioelectric resistance sensor. The vital functions of the animal are acquired by the first device with at least two first sensors and/or second sensors arranged at a distance from each other. In an embodiment, the at least two second sensors arranged at a distance from each other have measuring points that differ from those of the at least two first sensors arranged at a distance from each other.

In an embodiment, the at least two first and/or second sensors arranged at a distance from each other are secured to any position of the animal desired. In a preferred embodiment, the at least two first sensors arranged at a distance from each other are secured along the heart axis positions of the animal. In an embodiment, the heart axis position includes the anatomical and/or electrical heart axis position of the animal.

In an embodiment, the first and/or second sensors are fixedly cast in a casing. The casing here corresponds to a sleeve, sheath, capsule or housing. In an embodiment, the casing consists of plastic or glass. As a consequence, the casing is advantageously transparent to incoming and outgoing infrared light. In an alternative embodiment, the first and/or second sensors are detachably arranged in a casing.

In an embodiment, the first and/or second sensors measure and detect the corresponding data about vital functions wirelessly. In an embodiment, the first and/or second sensors measure via an infrared interface, i.e., the first and/or second sensors have both corresponding transmitters and receivers. The first and/or second sensors here emit light in the infrared range, which is reflected by the skin of the animal and thereby sent back to the first and/or second sensors, and there received with the correspondingly measured information. The geometry of the corresponding casings of the first and/or second sensors is here advantageously configured so that the infrared light generated by the sensor can propagate from the sensor in a conical or directed manner.

In another embodiment, the casing is here watertight and non-biodegradable. The first and/or second sensors are thus advantageously protected against environmental influences and the movements of the animal, which produce an additional mechanical load. The casing is advantageously transparent in design, so that the light emitted by the first and/or second sensors passes through the casing and hits the skin of the animal, and the signal reflected from there can pass through the casing and back into the sensor.

In an embodiment, given an electrical cable as the signal transmission means, the first and/or second sensors are detachably connected with the at least one signal transmission means. In an embodiment, the detachable connection is designed as a plug connection. The corresponding first and/or second sensors can advantageously be easily removed from the system for replacement and/or cleaning and/or repair purposes.

In a preferred embodiment, the system for measuring vital functions comprises at least one signal transmission means, which transmits the data measured by the first and/or second sensors to a data processing device. In another embodiment, the acquired data of the other modules of the second and/or third device are also transmitted to a data processing device by the signal transmission means.

In an embodiment, the at least one signal transmission means is designed as an electrical cable. In an alternative embodiment, the at least one signal transmission means is designed as a wireless connection, such as Bluetooth or WLAN.

In an embodiment, the at least one signal transmission means comprises a memory device. In an embodiment, for example, the memory device is designed as a flash memory or memory card, in particular as a microSD memory card, or as a USB stick, in particular a microUSB stick. The acquired data are advantageously stored on the memory device, and can be evaluated subsequently or at a later point. This is especially advantageous in situations in which the animal is located in regions with poor network coverage.

In another embodiment, the memory device is designed as a process-controlled memory unit, such as a data logger, in particular as a microSD data logger. As a consequence, the continuous storage of data and saving of measured data on a memory medium prevents a loss of data, for example as the result of a network failure or a temporarily unavailable network, making it impossible to transmit the data. The data can then be evaluated subsequently.

In another embodiment, the at least one signal transmission means comprises an audio data recording and playback device. In an embodiment, the audio recording and playback device plays back the input or output of voice commands or acoustic signals. This advantageously makes it possible to calm down the animal, or relay commands to the animal so as to train or lead it. In an embodiment, the audio data recording and playback device is designed as an MP3 recorder and player.

In another embodiment, the at least one signal transmission means comprises at least one light-emitting element. In another embodiment, the at least one light-emitting element serves as a readiness or malfunction indicator for the individual first and second sensors and modules of the system according to the invention. In an embodiment, for example, the at least one light- emitting element is designed as a lamp, in particular as an LED. The at least one light-emitting element allows the system user to display the status of the system. For example, this is effected by changing light colors of an LED.

In another embodiment, the at least one signal transmission means comprises an energy storage. In an embodiment, for example, the energy storage is designed as a charger, in particular as a battery or accumulator. In an embodiment, the data transmission intervals are controlled as a function of energy supply. The energy storage is advantageously monitored, and a situational deactivation of specific modules is enforced, for example so as to increase the runtime of the energy storage.

In an embodiment, the energy storage is connected with an energy generating option. In an embodiment, the energy generating option is designed as a solar module, which advantageously ensures an self-sufficient supply of the system according to the invention.

In a preferred embodiment, the system for acquiring the vital status further comprises a second device for determining the position and location of animals. The second device advantageously ensures an exact localization and geographical data transmission of the animal. As a consequence, the user is always informed about the current whereabouts of the animal.

In an embodiment, the second device for determining the position and location comprises a module for position determination, orientation and navigation. In an embodiment, for example, the module for position determination, orientation and navigation is designed as a GPS, GNSS or gyroscope. In an alternative embodiment, the position determination, orientation and navigation, in case of missing GPS connection, is designed as GSM localization via the mobile network, for example as cell tower triangulation by a GSM module. The GSM module can be designed as a SIM card, for example.

In another embodiment, the GNSS integrated into the module for position determination, orientation and navigation ensures that the speed of the animal can be determined.

In another embodiment, the GPS and GNSS integrated into the module for position determination, orientation and navigation further allows a height measurement (GPS leveling), which advantageously reflects the location of the animal as a function of its altitude.

In an embodiment, the second device for determining the position and location comprises a module for voice and data transmission. In an embodiment, for example, the module is designed as a GSM/3G connection. In an alternative embodiment, the module for voice and data transmission, in case of missing GSM/3G connection, is designed as a telecommunication service for transmitting text messages in the mobile network, e.g., SMS.

In an embodiment, the second device for determining the position and location of animals comprises a compass. The compass advantageously provides information about the direction in which the animal is moving.

In an embodiment, the second device for determining the position and location of animals comprises an optoelectronic detection means. The optoelectronic detection means advantageously makes it possible to visually monitor the environment of the animals, and hence determine the position. In an embodiment, the optoelectronic detection means is designed as a camera. In another embodiment, the optoelectronic detection means is designed as a video camera. The owner of the animal is advantageously given an impression of the environment of the animal in real time, without any visual contact with the animal. The environment includes both danger zones and landscape photographs.

In an embodiment, the second device for determining the position and location of animals comprises at least one light source. The light source advantageously serves to better illuminate the environment of the animal, which allows the animal to better orient itself, especially in a dark environment. In an embodiment, for example, the at least one light source is designed as a lamp, in particular as a LED.

In an embodiment, the module for determining the position, orientation and navigation designed as a GPS comprised of the second device or determining the position and location of animals is used to actuate a drone that independently follows the animal (for example, an AirDog drone, https:www.airdog.com/), so that the owner can also visually observe the precise whereabouts of the animal. As a result, animals used on rescue missions, such as rescue dogs, can be monitored when sent into areas that are normally no longer accessible, since there is no visual contact with the owner of the animal, so that the safety of the animal cannot be guaranteed.

In a preferred embodiment, the system comprises a third device selected from gas sensors and/or a module for positive owner identification, as well as a module for deactivation and blocking.

In an embodiment, the third device of the system comprises gas sensors. In conjunction with the drone, the animal trained for rescue missions can here also be sent under controlled observation into danger zones, such as fires, making it possible to measure the composition of the air for toxic gases. In an embodiment, the gas sensors are designed as CO₂ sensors.

In another embodiment, the third device of the system comprises a module for positive owner identification by means of ICCID, IMSI or serial numbers.

In another embodiment, the third device of the system comprises a module for deactivating and blocking the other modules. In an embodiment, this is done by way of a command function, an app or an alarm. This advantageously counteracts theft or third-party use.

In a preferred embodiment, the data about the vital functions of the animal are acquired by the second device and/or the third device.

In a preferred embodiment, the system for acquiring the vital status further comprises a positioning device.

In a preferred embodiment, the positioning device is designed as a stabilising device for medical treatment, for relieving strain, stabilising, securing and horizontally lifting and carrying animals. The positioning device preferably comprises at least one belly element and one back element, which can be detachably connected by connector elements.

In an embodiment, the positioning device has at least partially reflecting elements. The reflecting elements are preferably secured to the surface of the positioning device facing the environment, so that the wearer of the positioning device can be readily discerned by the reflection in road traffic, even in darkness or given poor lighting conditions.

In an embodiment, at least one component of the system for acquiring vital functions is arranged on the positioning device. In a preferred embodiment, the at least two first and/or second sensors arranged at a distance from each other and/or the at least one signal transmission means are arranged on the positioning device.

In a preferred embodiment, the first device with the at least two first and/or second sensors arranged at a distance from each other is introduced into the positioning device, either punctually or over the entire surface. In another embodiment, the positioning device incorporates the signal transmission means and/or the second device for determining the position and location and/or the third device in addition to the first device with the at least two first and/or second sensors arranged at a distance from each other.

In an embodiment, the positioning device has recesses for the at least two first and/or second sensors arranged at a distance from each other. The sensors can advantageously be pushed through these recesses, and the movement of the animals causes them to make their way up to the surface of their skin, where this established contact with the skin of the animal allows them to reliably record data. The first and/or second sensor here works its way through the fur and feathers of varying length up to the surface of the skin of the respective animal without leaving any bruises. Depending on the composition of the outer surface of the animal, it is also possible to select different shapes for the casings of the first and/or second sensors. For example, the casings could have a flattened, rounded or conical shape. The surface of the casing is smooth in design, so as not to cause any bruising or injuries to the animal. For attachment and latching purposes, the corresponding casing of the sensors here has a first disk, for example, which as the sensor is passed through the recess of the positioning device is in contact with the outwardly facing side of the positioning device, and fixed in place with a second counter-clamping disk, which is located on the interior side, i.e., on the side facing the animal, of the positioning device.

The at least two first and/or second sensors arranged at a distance from each other and secured to the positioning device in this way are advantageously in contact with the skin of the animal.

In an alternative embodiment, the at least two first and/or second sensors arranged at a distance from each other are introduced into the positioning device by weaving, knitting, braiding, bonding or pressing. This advantageously protects the first and/or second sensors against weather conditions.

In an embodiment, the optoelectronic detection means is secured to the positioning device. The image generated here is advantageously recorded at the height of the animal wearing the optoelectronic detection means, thereby providing the same viewing angle as the animal. This is relevant in particular with respect to rescue animals, such as rescue dogs, since they must press forward even in impassable terrain or caves, and the owner is in this way better informed than he or she would be if the optoelectronic detection means were secured to the positioning device as an attachment.

In an embodiment, the positioning device is at least partially flat in design, and has at least one flexible and anatomically adjustable supporting area. Anatomically adjustable here means that the positioning device flexibly clings to the body areas of the animal. This advantageously enhances wearing comfort. In addition, the design of the positioning device advantageously ensures that the muscles, joints and blood vessels of the animal are treated gently, for example. This preserves the musculoskeletal system of the animal.

In an embodiment, the positioning device runs along anatomically suitable positions of the animal. Anatomically suitable positions are to be understood as those positions that do not impede or hurt the animal while it moves, while still making it possible to advantageously measure the vital functions directly and in real time. By placing the positioning device on anatomically suitable positions of the animal, the varyingly designed fat structures and fat saturations in the skin of the animals are further advantageously taken into account, so that vital value functions can be precisely determined. In an embodiment, the positioning device runs along anatomically suitable positions of the rump of the animal. In an embodiment, the positioning device with the first and/or second sensors secured thereto runs over anatomically relevant body locations, such as along the heart axis positions, thereby advantageously resulting in a reliable measurement of the vital functions.

In an embodiment, the positioning device has a robust textile material. In an embodiment, the robust textile material has a water column of 3000 to 4000 mmH₂O, preferably 3500 mmH₂O. In another embodiment, the robust textile material of the stabilizing device has tensile strengths ranging from 1 to 5 kg/cm. The tensile of the robust textile material is advantageously selected as a function of the size of the dog, and hence of the size of the respective belly, chest, shoulder and back elements. The robust textile material of the positioning device is here designed so that the positioning device is dirt-repellant, washable and breathable.

In an embodiment, the positioning device is at least partially padded. In another embodiment, the positioning device at least partially has a temperature control device. In an embodiment, the temperature control device is heatable and/or coolable in design.

In a preferred embodiment, the positioning device is designed as a commercially available harness. In another embodiment, the positioning device is designed as a commercially available belt system. In another embodiment, the positioning device is designed as a commercially available belt strap.

In an embodiment, the at least one signal transmission means, if designed as an electrical cable, is permanently installed in the positioning device. The signal transmission means here runs in particular along the edge of the positioning device. In an embodiment, the edge of the positioning device is made out of a stable material. In an embodiment, the edge of the positioning device is made out of leather, such as genuine or imitation leather, or neoprene. The at least one signal transmission means is thus outwardly protected in the positioning device. As a result, the positioning device can advantageously be washed without having to remove the signal transmission means beforehand.

In an embodiment, the positioning device can be individually configured and combined in variable and differing dimensions, depending on the size of the animal. Attention is here advantageously paid to the variability of body composition, such as size and weight, given the wide variety of animal species. The varying physiques of the animals results in correspondingly different vital functions, such as highly variable pulse and respiration rates.

In a preferred embodiment, the components of the system for acquiring vital functions are designed so that they can be detachably connected with the positioning device by connectors. In another embodiment, in particular the first and/or second sensors are here designed so that they can be detachably connected with the positioning device by connectors. In an embodiment, the connectors comprise mechanical connectors, such as buckles, in particular roller buckles, double pin buckles, triple pin buckles, ladder-lock buckles, sliding buckles, clamping buckles, click buckles (quick fit, also called click clasps), push-in buckles or interlocking buckles. In another embodiment, the connectors comprise mechanical connectors, such as toggle closures, hook and loop closures, press studs or zip fasteners. The components of the system for acquiring vital functions are hence advantageously secured to the positioning device in a stable manner, and can be easily removed from the latter or replaced.

In a preferred embodiment, the data about the vital functions of the animal measured by the first and/or second sensors of the first device are transmitted by the signal transmission means to a data processing device, and there processed and evaluated. For example, a performance curve is generated for the activities of the animal. In another embodiment, the data about the animals measured by the second device and/or third device are transmitted by the signal transmission device to a data processing device, and there processed and evaluated.

In an embodiment, the data processing device comprises a processor, which is connected by various signal lines with other elements of the data processing device, such as a working memory or a GSM module for transmitting data in a cloud.

In an alternative embodiment, the data processing device is designed as a cloud. The data measured by the first and/or second sensors are advantageously sent to the cloud by signal transmission means, which are wireless in design. As a consequence, the user can retrieve the status of the animal along with its position in real time.

In an embodiment, the system for determining the vital status of animals further comprises a housing for accommodating components of the system, as well as for protecting against mechanical influences. At least a part of the system components is secured in the housing. In an embodiment, the housing incorporates at least parts of the signal transmission means and/or at least parts of the first device for acquiring the vital functions and/or at least parts of the second device for determining the position and location and/or at least parts of the third device. In an embodiment, the housing incorporates the energy storage and/or the memory device and/or light-emitting elements and/or the GSM module and/or the compass and/or the working memory.

In an embodiment, the housing is arranged on the exterior side of the positioning device. Arrangement here takes place with quickly detachable connections, such as snap connections, hook and loop closeners or even screw connections. This advantageously allows it to be easily and quickly removed from the positioning device by the user.

In an embodiment, the housing is rigid and/or watertight and/or stable in design. The components contained therein are advantageously protected against environmental influences and shocks. In another embodiment, the housing is non-biodegradable in design. In another embodiment, externally arising vibrations are attenuated by the configuration of the housing, thereby damping the housing.

The individual housing parts are here interconnected by sealings. The housing preferably has a two-part design. The housing is advantageously easy to open, so that the corresponding components contained therein can be changed out and introduced for retrofitting in the event of defects or for repair purposes. In an embodiment, the housing consists of more than one part.

In an embodiment, the housing consists of an epoxy resin. The housing is advantageously comprised of a transparent epoxy resin, thereby ensuring inside views of the system components contained therein. In particular when light-emitting elements are arranged in the housing, users of the system can have the status of the system displayed to them.

In a preferred embodiment, the data about the vital functions of the animal measured by the first device are transmitted by the data processing device to an external output device. In a preferred embodiment, the external output device outputs the vital status of animals. In another embodiment, the external output device outputs the data about position and location of the second device. In another embodiment, the external output device outputs the data acquired by the third device.

In another embodiment, the data about vital functions are transmitted by the data processing device to the external output device via a wireless connection, such as Bluetooth or WLAN.

In an embodiment, the external output device is designed as a computer, tablet, laptop or smartphone. A user-friendly operation of the external output device advantageously takes place via specially programmed apps. In another embodiment, the data about vital functions are visualized by the external output device, and displayed as overviews and statistics.

In an embodiment, the external output device has a computer program product, which evaluates the data about vital functions. The activity, general condition and health of the animal are here advantageously considered.

In an embodiment, the external output device emits a warning signal if values exceed or drop below standard levels for vital functions. As a consequence, the animal is advantageously monitored in real time, thereby enabling a timely intervention, for example involving the movement or food intake of the animal.

In a preferred embodiment, the external output device is designed to send back commands to the data processing device, which in turn are transmitted to the first and/or second sensors.

In an embodiment, the acquired data are transmitted from the second device and/or third device to a data processing device by at least one signal transmission means, processed and evaluated, and transmitted from the data processing device to an external output device, which outputs the vital status of animals.

Depending on the need and situation, the modular composition of the system advantageously makes it possible to enable or disable individual modules for data acquisition. As a result, energy can be saved, and undesired, redundantly collected data can be avoided. If the owner of the animal wants to use the system only for determining the location of the animal, for example, he or she does not need the first device for acquiring vital functions.

For example, the data about vital functions acquired by the at least two first and/or second sensors arranged at a distance from each other are of interest to the owner of the animal in that he or she can take certain stress factors into account while interacting with the animal. While riding a bicycle, for example, the owner of a dog can observe the load on the dog running next to the bicycle in real time, and estimate when the dog has reached its limit.

In addition, the data about vital functions acquired by the at least two first and/or second sensors arranged at a distance from each other are also of interest to veterinarians and pharmaceutical manufacturers, and for veterinary medicine studies and investigations. The system according to the invention can advantageously be used to investigate the energy balance of the animal. Providing the veterinarian with additional information about the behavior of the animal, for example when and what the animal eats and/or drinks makes it possible, in conjunction with the measured data about vital functions, to assess the continued handling of the animal. Additional interesting actual values, such as overviews regarding the movement times of the animal or medication intake, can also be drawn upon for evaluating and providing better treatment or therapy to the animal.

In an embodiment, the system according to the invention for acquiring the vital status ensures that the wellbeing of the animal is monitored. The owner of the animal is advantageously informed in real time about the health, and hence condition, of the animal. Arising stress factors reflected in the data about vital functions, for example an elevated heart or respiratory rate, and thus values that exceed or drop below standard levels, can here be analyzed and evaluated, making it possible to individually treat the animal.

For the purpose of realising the invention, it is also beneficial to combine the variants and embodiments according the invention described previously and the features of the claims in any order.

### EXEMPLARY EMBODIMENTS

In the following text, the invention will be explained in greater detail on the basis of an exemplary embodiment. The embodiment relates to a stabilising device for dogs and is intended to describe the invention without any limitation thereof.

The invention will be explained in greater detail with reference to drawings. The drawing shows:

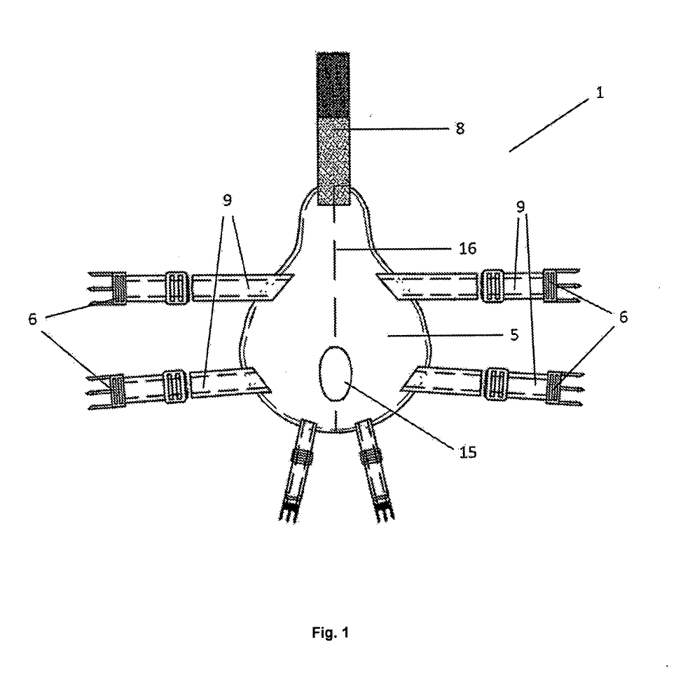

FIG. 1 Belly element of the system for acquiring the vital functions of animals,

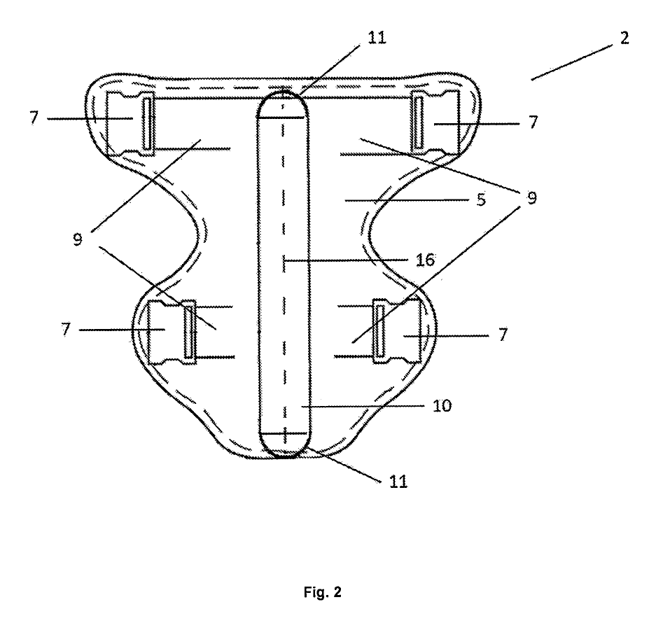

FIG. 2 Back element of the system for acquiring the vital functions of animals,

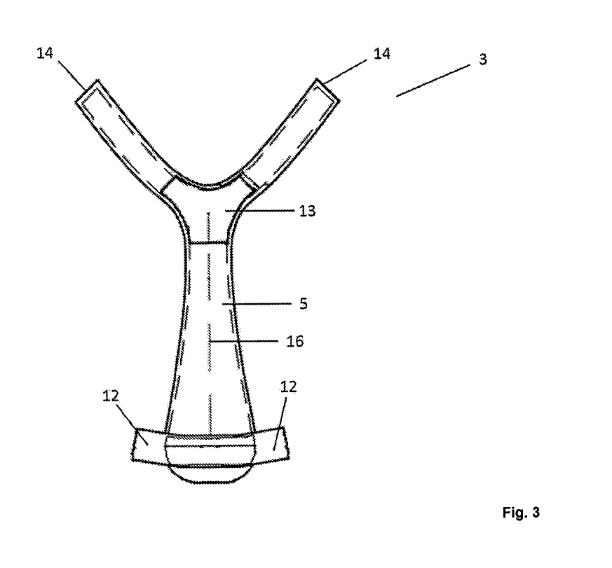

FIG. 3 Chest element of the system for acquiring the vital functions of animals,

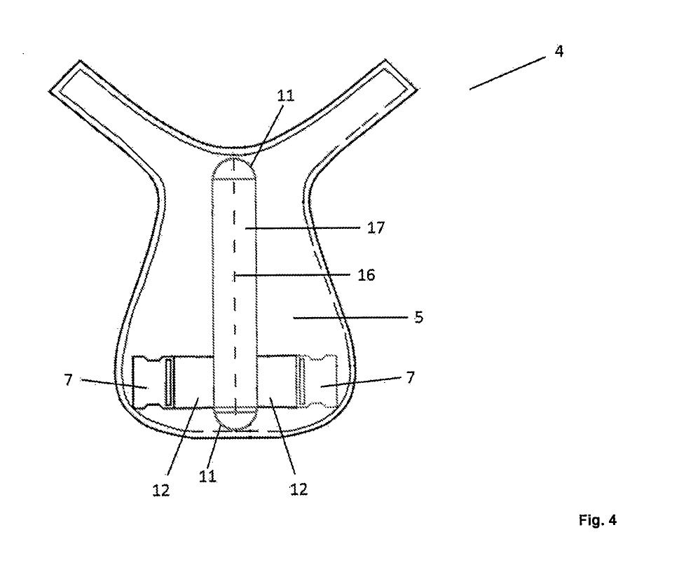

FIG. 4 Shoulder element of the system for acquiring the vital functions of animals,

The upper parts of the components depicted on the figures each point toward the head of the dog, while the lower parts of the components point toward the tail of the dog. The respectively longitudinal axes of the components recorded from the top down run along the spine of the dog, and relate to its running direction.

FIG. 1 shows a belly element 1, which has a pear-shaped supporting area 5.

The belly element 1 has four belt straps 9 arranged punctually thereon, which are arranged mirror symmetrically to the longitudinal axis 16 of belly element 1 and at a distance from each other. Two belt straps 9 are arranged on the left side and two belt straps 9 are arranged on the right side of supporting area 5 of the belly element 1 relative to the longitudinal axis 16. In addition, the four punctually arranged belt straps 9, two narrower belt straps 9 are shown on the lower side of the belly element 1 in the figure, through which the dog's tail is guided. The length of all belt straps 9 can be adjusted with sliding bars.

In proximity to the straps 9 arranged on the stomach element 1, holes (not shown on the figure) are provided in the supporting surface 5, which have a diameter of approx. 1 cm. These holes are suitable for guiding through sensors.

The cutout 15 to allow the sex organ of a male dog to guide through is provided centrally and symmetrically to the longitudinal axis 16 in the belly element 1.

The belt straps 9 are sewn to the belly element. A click buckles 6 is provided on each belt strap 9 through which the belly element 1 is connected to the back element 2 (connector and click buckles not shown in the figure).

The top of the belly element 1 is equipped with a connecting strap 8 as a detachable connecting means which extends parallel to the longitudinal axis 16 and by which the belly element 1 is connected to a chest element 3 (chest element and connector not shown in the figure). The connecting strap 8 is sewn to the belly element 1 in the same way as the belt straps 9.

The belly element 1 is padded and includes a spacer fabric (not shown in the figure).

FIG. 2 shows a back element 2 with a flat supporting area 5. The back element 2 has four belt straps 9, which are sewn onto the back element 2 parallel, perpendicular and at a distance from each other relative to the longitudinal axis 16 and arranged mirror symmetrically to the longitudinal axis 16. A counterpiece 7 for each click buckle 6 attached to the belly element 1 is arranged on each belt strap 9, and by which the belly element 1 is detachably connected to the back element 2 (connector not shown in the figure).

A back brace 10 made of plastic is fastened to the back element 2, and is sewn underneath the belt straps 9 which extend over the back brace 10 of the back element 2. The back brace 10 extends continuously along the longitudinal axis 16 of the back element 2, and thus also along the dog's spine as far as to the head of its tail.

The back element 2 further has a D-ring 11 at both its upper and lower ends along the longitudinal axis 16. The D-rings 11 are each sewn to a belt strap 9 at the two most distant points of the back element 2 along the longitudinal axis 16. The end of a carrying handle may be clipped into each D-ring 11 (not shown in the figure).

The back element 2 is padded (not shown in the figure).

FIG. 3 shows a chest element 3 which has a supporting area 5 in the shape of a tuning fork.

The bottom of the chest element 3 has two belt straps 12 arranged punctually on the chest element 3, each of which is arranged mirror symmetrically to the longitudinal axis 16 of the chest element 3. The belt straps 12 extend continuously over the chest element 3. One click buckle (not shown in the figure) arranged thereon is attached to each belt strap 12.

The chest element 3 has a detachable connector 14 at the ends of each of the two tines of the tuning fork-shaped supporting area 5, by which connectors the chest element 3 is detachably connected with the shoulder element 4 (shoulder element not shown in the figure).

The bottom of the chest element 3 has a hook and loop closure for the connecting strap 8 coming from the belly element 1, by which the chest element 3 is connected to a belly element 1 (connecting strap and hook and loop closure not shown in the figure). The connecting strap 8 is also sewn to the belly element 1 in the same way as the belt straps (9 and 12).

The chest element 3 has a sternum padding 13 and a spacer fabric (spacer fabric not shown in the figure).

FIG. 4 shows a shoulder element 4, which has a tuning fork-shaped supporting area 5 which is wider in design than the surface of the chest element 3.

The bottom of the shoulder element 4 has two belt straps 12 arranged punctually on the shoulder element 4, each of which is arranged mirror symmetrically to the longitudinal axis 16 of the shoulder element 4. The belt straps 12 extend continuously over the shoulder element 4. A counterpiece 7 for each click buckle 6 attached to the chest element 3 is arranged on each belt strap 12, and by which the chest element 3 is detachably connected to the shoulder element 4 (chest element and connector not shown in the figure).

The shoulder element further has a click closure at the ends of its two tines, by which the shoulder element is connected detachably to the chest element (not shown in the figure).

A shoulder brace 17 made of plastic is fastened to the shoulder element 4, and is sewn underneath the belt straps 12 which extend over the shoulder brace 17 of the shoulder element 4. The shoulder brace 17 extends continuously along the longitudinal axis 16 of the shoulder element 4 and thus also along the dog's spine.

The shoulder element 4 further has a D-ring 11 at both its upper and lower ends along the longitudinal axis 16. The D-rings 11 are each sewn to a belt strap 12 at the two most distant points of the shoulder element 4 along the longitudinal axis 16. The end of a carrying handle may be clipped into each D-ring 11 (not shown in the figure). The two ends of the carrying handle may also be clipped into a D-ring on the shoulder element 4 and a D-ring 11 on the back element 2, so that the dog may be carried both by the shoulder element 4 and by the back element 2.

The shoulder element 4 is padded (not shown in the figure).

### REFERENCE SIGNS

1 Belly element
2 Back element
3 Chest element
4 Shoulder element
5 Supporting area
6 Click buckle
7 Click buckle counterpiece
8 Detachable connecting strap (on the belly element) with the chest element
9 Detachable belt strap (on the belly element) with the back element
10 Back brace
11 D-rings for attaching carrying handles
12 Detachable belt strap (on the chest element) with the shoulder element
13 Sternum padding
14 Detachable connector (on the chest element) with the shoulder element
15 Cutout for a male dog's sex organs
16 Longitudinal axis
17 Shoulder brace

1. A system for acquiring the vital status of animals, comprising:
at least one first device for acquiring vital functions, and
at least one signal transmission means,
wherein the first device has at least two first sensors arranged at a distance from each other.

2. The system according to claim 1, characterized in that the at least two first sensors are arranged at a distance from each other are designed as a pulse oximeter and/or body temperature sensor and/or temperature sensor and/or pedometer and/or bioelectric resistance sensor.

3. The system according to claim 1, characterized in that the first device further has at least two second sensors arranged at a distance from each other, which are different than the at least two first sensors arranged at a distance from each other, wherein the first sensors and second sensors are selected from a pulse oximeter and/or body temperature sensor and/or temperature sensor and/or pedometer and/or bioelectric resistance sensor.

4. The system according to claim 1, characterized in that it further comprises a second device for determining the position and location of animals.

5. The system according to claim 1, characterized in that it further comprises a third device selected from gas sensors and/or a module for positive owner identification and/or a module for deactivation and blocking.

6. The system according to claim 1, further comprising a positioning device, on which at least two first and/or second sensors are arranged at a distance from each other and/or the at least one signal transmission means are arranged.

7. The system according to claim 1, characterized in that the at least two first and/or second sensors are arranged at a distance from each other are incorporated into the positioning device, either punctually or over the entire surface.

8. The system according to claim 1, characterized in that the at least two first and/or second sensors are arranged at a distance from each other are attached along the heart axis positions of the animal.

9. The system according to claim 1, characterized in that the components of the system for acquiring the vital functions of animals can be detachably connected with the positioning device by connectors.

10. The system according claim 1, characterized in that the positioning device is designed as a stabilising device for medical treatment, relieving strain, stabilising, securing and horizontally lifting and carrying animals, comprising at least one belly element and one back element, which can be detachably connected by connector elements.

11. A method for acquiring the vital status of animals, wherein the vital functions are acquired by a first device with at least two first sensors arranged at a distance from each other, wherein the acquired data are transmitted by at least one signal transmission means to a data processing device, and there processed and evaluated, and from the data processing device to an external output device, which outputs the vital status of animals.

12. The method according to claim 11, characterized in that the data about the vital functions are acquired by at least two first sensors arranged at a distance from each other and/or two second sensors arranged at a distance from each other.

13. The method according to claim 11, characterized in that data about the vital functions are acquired by a second device and/or a third device.

14. Use of a system for acquiring the vital status of animals according to claim 1.

15. Use of a method for determining the vital status of animals according to claim 11.
